(19) United States
(12) Patent Application Publication
(10) Pub. No.: US 2010/0291163 A1
(43) Pub. Date: Nov. 18, 2010

(54) ORAL CARE COMPOSITION COMPRISING NANOPARTICULATE TITANIUM DIOXIDE

(76) Inventor: Christabel Fowler, Surrey (GB)

Correspondence Address: GlaxoSmithKline;GLOBAL PATENTS -US, UW2220, P. O. BOX 1539, KING OF PRUSSIA, PA, 19406-0939, US

(21) Appl. No.: 12/438,070

(22) PCT Filed: Aug. 22, 2007
(86) PCT No.: PCT/EP2007/058746

§ 371 Date: May 7, 2010

(30) Foreign Application Priority Data
Aug. 24, 2006 (GB) 0616823.1
Apr. 5, 2007 (GB) 0706780.4

(51) Int. Cl. A61K 8/81; A61Q 11/00

(52) U.S. Cl. 424/401; 424/52; 424/49

(57) ABSTRACT

The present invention relates to an oral care composition comprising nanoparticulate titanium dioxide, optionally together with a source of fluoride ions, for combating dental erosion and/or tooth wear. In addition such compositions may also have benefit in tooth whitening. When a source of fluoride ions is present such compositions are also of benefit in combating dental caries.

The present invention relates to an oral care composition comprising nanoparticulate titanium dioxide, optionally together with a source of fluoride ions, for combating (i.e. helping to prevent, inhibit and/or treat) dental erosion and/or tooth wear. In addition such compositions may also have benefit in tooth whitening. When a source of fluoride ions is present such compositions are also of benefit in combating dental caries.

Tooth mineral is composed predominantly of calcium hydroxyapatite, Ca₁₀(PO₄)₆(OH)₂, which may be partially substituted with anions such as carbonate, or fluoride, and cations such as zinc or magnesium. Tooth mineral may also contain non-apatitic mineral phases such as octacalcium phosphate and calcium carbonate.

Tooth loss may occur as a result of dental caries, which is a multifactorial disease where bacterial acids such as lactic acid produce sub-surface demineralisation that does not fully remineralise, resulting in progressive tissue loss and eventually cavity formation. The presence of a plaque biofilm is a prerequisite for dental caries, and acidogenic bacteria such as Streptococcus mutans may become pathogenic when levels of easily fermentable carbohydrate, such as sucrose, are elevated for extended periods of time.

Even in the absence of disease, loss of dental hard tissues can occur as a result of acid erosion and/or physical tooth wear; these processes are believed to act synergistically. Exposure of the dental hard tissues to acid causes demineralisation, resulting in surface softening and a decrease in mineral density. Under normal physiological conditions, demineralised tissues self-repair through the remineralising effects of saliva. Saliva is supersaturated with respect to calcium and phosphate, and in healthy individuals saliva secretion serves to wash out the acid challenge, and raises the pH so as to alter the equilibrium in favour of mineral deposition.

Dental erosion (i.e. acid erosion or acid wear) is a surface phenomenon that involves demineralisation, and ultimately complete dissolution of the tooth surface by acids that are not of bacterial origin. Most commonly the acid will be of dietary origin, such as citric acid from fruit or carbonated drinks, phosphoric acid from cola drinks and acetic acid such as from vinaigrette. Dental erosion may also be caused by repeated contact with hydrochloric acid (HCl) produced by the stomach, which may enter the oral cavity through an involuntary response such as gastroesophageal reflux, or through an induced response as may be encountered in sufferers of bulimia.

Tooth wear (i.e. physical tooth wear) is caused by attrition and/or abrasion. Attrition occurs when tooth surfaces rub against each other, a form of two-body wear. An often dramatic example is that observed in subjects with bruxism, a grinding habit where the applied forces are high, and is characterised by accelerated wear, particularly on the occlusal surfaces. Abrasion typically occurs as a result of three-body wear and the most common example is that associated with brushing with a toothpaste. In the case of fully mineralised enamel, levels of wear caused by commercially available toothpastes are minimal and of little or no clinical consequence. However, if enamel has been demineralised and softened by exposure to an erosive challenge, the enamel becomes more susceptible to tooth wear. Dentine is much softer than enamel and consequently is more susceptible to wear. Subjects with exposed dentine should avoid the use of highly abrasive toothpastes, such as those based on alumina. Again, softening of dentine by an erosive challenge will increase susceptibility of the tissue to wear.

Dentine is a vital tissue that in vivo is normally covered by enamel or cementum depending on the location i.e. crown versus root respectively. Dentine has a much higher organic content than enamel and its structure is characterised by the presence of fluid-filled tubules that run from the surface of the dentine-enamel or dentine-cementum junction to the odontoblast/pulp interface. It is widely accepted that the origins of dentine hypersensitivity relate to changes in fluid flow in exposed tubules, (the hydrodynamic theory), that result in stimulation of mechanoreceptors thought to be located close to the odontoblast/pulp interface. Not all exposed dentine is sensitive since it is generally covered with a smear layer; an occlusive mixture comprised predominantly of mineral and proteins derived from dentine itself, but also containing organic components from saliva. Over time, the lumen of the tubule may become progressively occluded with mineralised tissue. The formation of reparative dentine in response to trauma or chemical irritation of the pulp is also well documented. Nonetheless, an erosive challenge can remove the smear layer and tubule “plugs” causing outward dentinal fluid flow, making the dentine much more susceptible to external stimuli such as hot, cold and pressure. As previously indicated, an erosive challenge can also make the dentine surface much more susceptible to wear. In addition, dentine hypersensitivity worsens as the diameter of the exposed tubules increases, and since the tubule diameter increases as one proceeds in the direction of the odontoblast/pulp interface, progressive dentine wear can result in an increase in hypersensitivity, especially in cases where dentine wear is rapid.

Loss of the protective enamel layer through erosion and/or acid-mediated wear will expose the underlying dentine, and are therefore primary aetiological factors in the development of dentine hypersensitivity.

It has been claimed that an increased intake of dietary acids, and a move away from formalised meal times, has been accompanied by a rise in the incidence of dental erosion and tooth wear. In view of this, oral care compositions which help prevent dental erosion and tooth wear would be advantageous.

WO 00/59460 (Grace) relates to porous inorganic oxide-based dentifrice additives with particle size in the range 0.05 to 3 microns, for use in tooth sensitivity and remineralisation. Examples of inorganic oxide particles include SiO₂, Al₂O₃, MgO, TiO₂ and ZrO₂.

WO 02/051945 (Henkel) relates to nanoparticulate titanium dioxide with a mean particle diameter ranging from 10 to 1000 nm being coated with a polar organic surface-modifying agent. The particles are described as being suitable as tooth-brightening agents. Suitable surface-modifying agents include substances containing at least one functional group selected from carboxy, sulphono, phosphono, isocyanoto, hydroxy, amino, or an epoxy group and various silanes. Preferred surface-modifying agents include substances containing two or more functional groups selected from carboxylic acids, phosphonic acids, amino acids, sulphonic acids and certain silanes.

There is no suggestion in the above-noted documents that the inorganic oxides have any benefit or utility in protecting dental enamel from acid erosion and/or tooth wear.

The present invention is based on the discovery that nanoparticulate titanium dioxide strengthens and hardens dental enamel thereby providing protection against dental erosion and/or tooth wear.

Accordingly, in a first aspect the present invention provides the use of nanoparticulate titanium dioxide in the manufacture of an oral care composition for combating dental erosion and/or toothwear.

The titanium dioxide may be uncoated or may be surface-coated.

Suitably the titanium dioxide is surface-coated with a material that enhances its substantivity to the tooth (enamel and dentine) surface. Suitably such surface coating material also acts as a dispersing agent which when mixed with a suspension of uncoated nanoparticles can adsorb onto their surface to provide steric or ionic barriers so to help prevent their agglomeration or aggregation.

Examples of such a surface-coating material include a polyol or polyvinylpyrrolidone or a derivative thereof.

In a further aspect, the present invention provides an oral care composition comprising nanoparticulate titanium dioxide surface-coated with a polyol or polyvinylpyrrolidone (PVP) or a derivative thereof and an orally acceptable carrier or excipient.

In addition to combating dental erosion and/or toothwear such compositions may be of use in whitening teeth.

Suitably the surface-coating material is a polyol, which is a polyhydric alcohol, selected from the group consisting of glycerin (glycerol), propylene glycol, polyethylene glycol, polyvinyl alcohol, sorbitol, mannitol or xylitol or a mixture thereof.

Suitably the surface-coating material is PVP or a derivative thereof including, vinylpyrrolidine vinyl acetate copolymer (VP/VA) or vinylpyrrolidone vinyl alcohol (VP/VOH) copolymer or a mixture thereof.

Suitably the nanoparticulate titanium dioxide is surface-coated with glycerin or propylene glycol.

Suitably the nanoparticulate titanium dioxide is surface-coated with PVP.

Surface-coating may be achieved by covalent bonding of the coating material to the titanium dioxide or by electrostatic means.

Suitably a suspension of uncoated nanoparticulate titanium dioxide may be mixed with a solution of the surface coating material to provide a stabilised dispersion of the coated nanoparticles which can be used directly, or the coated nanoparticles can be isolated and then subsequently used, in the preparation of compositions of the present invention.

Suitably, the uncoated or surface-coated nanoparticulate titanium dioxide for use in compositions of the present invention has a mean particle diameter in the range from 2 nm to 500 nm, more suitably from 5 nm to 250 nm.

Compositions of the present invention suitably comprise between 0.25 and 20 % w/w of nanoparticulate titanium dioxide, for example between 0.5 and 10% w/w.

Surface-coating of the nanoparticulate titanium dioxide has the advantage of improving particle substantivity to the tooth surface, thereby promoting film formation, increasing the adhesive interaction and extending the duration of anti-erosion and/or tooth wear behaviour.

Compositions of the present invention may further comprise a dispersing agent which can adsorb onto the surface of the coated or uncoated nanoparticles to provide steric or ionic barriers so to help prevent their agglomeration or aggregation. Suitable dispersing agents are surfactants including solubilising or wetting agents or water-soluble polymers such as polyelectrolytes.

Compositions of the present invention may further comprise a source of soluble fluoride ions such as those provided by an alkali metal fluoride such as sodium fluoride, an alkali metal monofluorophosphate such a sodium monofluorophosphate, stannous fluoride, or an amine fluoride in an amount to provide from 25 to 3500 pm of fluoride ions, preferably from 100 to 1500 ppm. A suitable fluoride source is an alkali metal fluoride such as sodium fluoride, for example the composition may contain 0.1 to 0.5% by weight of sodium fluoride, eg 0.205% by weight (equating to 927 ppm of fluoride ions), 0.2542% by weight (equating to 1150 ppm of fluoride ions) or 0.315% by weight (equating to 1426 ppm of fluoride ions).

Fluoride ions enhance remineralisation and decrease demineralisation of dental enamel and are of benefit in combating caries and/or dental erosion.

In order to treat dentinal hypersensitivity the oral compositions of the present invention suitably further comprise a desensitising amount of a desensitising agent. Examples of desensitising agents include a tubule blocking agent or a nerve desensitising agent and mixtures thereof, for example as described in WO 02/15809. Suitable desensitising agents include a strontium salt such as strontium chloride, strontium acetate or strontium nitrate or a potassium salt such as potassium citrate, potassium chloride, potassium bicarbonate, potassium gluconate and especially potassium nitrate.

Compositions of the present invention will contain appropriate formulating agents such as abrasives, surfactants, thickening agents, humectants, flavouring agents, sweetening agents, opacifying or colouring agents, preservatives and water, selected from those conventionally used in the oral care composition art for such purposes. Examples of such agents are as described in EP 929287.

The oral compositions of the present invention are typically formulated in the form of toothpastes, sprays, mouthwashes, gels, lozenges, chewing gums, tablets, pastilles, instant powders, oral strips and buccal patches.

The compositions according to the present invention may be prepared by admixing the ingredients in the appropriate relative amounts in any order that is convenient and if necessary adjusting the pH to give a desired value.

In a further aspect the uncoated or coated nanoparticulate titanium dioxide may be incorporated into a dentifrice composition of the type described in WO2006/100071, the contents of which are incorporated herein by reference.

Accordingly the present invention further provides a dentifrice composition which composition comprises nanoparticulate titanium dioxide as hereinbefore described, a fluoride ion source as hereinbefore described and a silica dental abrasive, the dentifrice having a Relative Dentine Abrasivity (RDA) value from 20 to 60 and a pH in the range 6.5 to 7.5 and being free of an orthophosphate buffer or a water-soluble salt of a C₁₀₋₁₈ alkyl sulphate.

The pH referred to is that measured when the dentifrice composition is slurried with water in a 1:3 weight ratio of the composition to water.

Suitably the nanoparticulate titanium dioxide is formulated together with a dispersing agent as hereinbefore described.

Suitably the dentifrice composition of the present invention does not include a calcium salt which can reduce the availability of free fluoride ions.

Examples of suitable silica dental abrasives include those marketed under the following trade names Zeodent, Sident, Sorbosil or Tixosil by Huber, Degussa, Ineos and Rhodia respectively. The silica abrasive should be present in an amount sufficient to ensure the RDA of the dentifrice is between 20 and 60, for example between 25 and 50 or between 25 and 40 to ensure adequate cleaning of teeth by the dentifrice whilst not promoting abrasion of teeth, especially teeth suffering from dental erosion or having been softened by an acidic challenge.

The silica abrasive is generally present in an amount up to 15% by weight of the total composition, for example from 2 to 10% by weight, generally at least 5% for example from 5 to 7% by weight, suitably 6% by weight of the total composition. Reducing the level of silica abrasive has the advantage of not only lowering the abrasivity of the dentifrice but also minimising any interaction of the abrasive (or trace amounts of contaminants in the abrasive) with fluoride ions thereby increasing the availability of free fluoride ions.

Suitable surfactants for use in the dentifrice composition of the present invention include amphoteric surfactants for example, long chain alkyl betaines, such as the product marketed under the tradename ‘Empigen BB’ by Albright & Wilson, and preferably long chain alkyl amidoalkyl betaines, such as cocamidopropylbetaine, or low ionic surfactants such as sodium methyl cocoyl taurate, which is marketed under the trade name Adinol CT by Croda, or mixtures thereof. An amphoteric surfactant can be used alone as sole surfactant or can be combined with a low ionic surfactant.

Suitably, the surfactant is present in the range 0.1 to 10%, for example 0.1 to 5% such as from 0.5 to 1.5% by weight of the total composition.

Suitable thickening agents include, for instance, nonionic thickening agents such as, for example, (C1-6)alkylcellulose ethers, for instance methylcellulose; hydroxy(C1-6)alkylcellulose ethers, for instance hydroxyethylcellulose and hydroxypropylcellulose; (C2-6)alkylene oxide modified (C1-6)alkylcellulose ethers, for instance hydroxypropyl methylcellulose; and mixtures thereof. Other thickening agents such as natural and synthetic gums or gum like material such as Irish Moss, xanthan gum, gum tragacanth, carrageenan, sodium carboxymethylcellulose, polyvinylpyrrolidone, polyacrylic acid polymer (carbomer), starch and thickening silicas may also be used. Suitably the thickening agent is mixture of a thickening silica and xanthan gum, optionally with carrageenan and/or a carbomer.

Suitably the thickening agent is present in the range 0.1 to 30%, for example from 1 to 20%, such as from 5 to 15% by weight of the total composition.

Suitable humectants for use in compositions of the invention include for instance, glycerin, xylitol, sorbitol, propylene glycol or polyethylene glycol, or mixtures thereof; which humectant may be present in the range from 10 to 80%, for example from 20 to 60%, such as from 25 to 50% by weight of the total composition.

In order to combat dentine hypersensitivity the dentifrice composition of the present invention may further comprise a desensitising agent as hereinbefore described, especially potassium nitrate. The presence of potassium nitrate advantageously may provide an enhanced stain removal effect, which is of particular benefit for low abrasivity formulations, which otherwise might be expected to have relatively low cleaning performance.

The pH of the dentifrice composition of the present invention is in the range 6.5 to 7.5, suitably from 6.8 to 7.2, for example 7.1 and can be adjusted by the incorporation of a base such as sodium hydroxide.

In a further aspect the present invention also provides another dentifrice composition comprising nanoparticulate titanium dioxide as hereinbefore described, a fluoride ion source as hereinbefore described (for example an alkali metal fluoride), a thickening system comprising a thickening silica in combination with xanthan gum optionally with carrageenan and/or a carbomer, an anionic surfactant (for example a water-soluble salt of a C₁₀₋₁₈ alkyl sulphate such as sodium lauryl sulphate) and a silica dental abrasive in an amount up to 20% (suitably from 5 to 20% for example from 10 to 16%) by weight of the total composition, the dentifrice having a pH in the range from 6.0 to 8.0 (for example from 6.5 to 7.5), and being free of an orthophosphate buffer or a calcium salt. If desired such a dentifrice composition may also comprise a desensitising agent as hereinbefore described.

The present invention also provides a method of combating dental erosion and/or tooth wear which comprises applying an effective amount of a composition comprising nanoparticulate titanium dioxide as hereinbefore defined to an individual in need thereof.

The invention is further illustrated by the following Examples.

### EXAMPLE 1

### Microindentation as a Measure of Enamel Hardness

Human enamel chips were mounted in acrylic resin and polished flat using silicon carbide paper (1200 and 2400 grit). The specimens were then randomised and divided into three treatment groups (n=6). The treatment groups were 300 ppm fluoride (sodium fluoride); glycerin-coated titanium dioxide (with a mean particle size of 20 nm) aqueous suspension, 2.5% w/v (UV Titan M212, Kemira, Aston Chemicals); and deionised water. The baseline hardness of each specimen was determined using a Struers Duramin Microindentor, fitted with a Vickers diamond indenter. Hardness values were expressed as Vickers Hardness Numbers (VHN). A load of 1.961N was applied to the specimens, with a dwell time of 20 seconds.

Specimens were placed in 30 ml of one of the three test solutions with agitation for 120 seconds, before rinsing with deionised water. After treatment, microhardness measurements were repeated. Erosion was then performed by incubating the mounted specimens for 30 minutes in 10 ml of a 1.0% w/w solution of citric acid, pH 3.8. Specimens were removed from the erosive challenge at 10 minute intervals and the surface microhardness determined.

Scanning electron microscopy was carried out on human enamel specimens previously incubated in a 2.5% w/v glycerin-coated titanium dioxide aqueous suspension, a 2.5% w/v standard micron-sized titanium dioxide aqueous suspension, and water alone, Energy Dispersive X-ray Analysis (EDX), was used to identify titanium on the surface of the enamel after washing with water under flow for one minute.

### RESULTS

The results of the softening study are summarised in FIG. 1. The values for enamel hardness have been normalised relative to the individual baseline microhardness values, thus data at subsequent time points reflects softening of the enamel. The error bars in FIG. 1 represent standard deviations.

All the enamel specimens treated with 300 ppm fluoride or water softened during the period in which they were exposed to citric acid, increasingly so as the incubation time increased. Specimens treated with the nanoparticle suspension did not soften significantly during the first 10 mins exposure to citric acid. After 20 and 30 mins incubation in the acid, enamel treated with fluoride or the titanium dioxide nanoparticle suspension softened significantly less than enamel treated with water. After 20 mins citric acid exposure, specimens treated with fluoride or the nanoparticle suspension were equivalent in their extent of softening. After 30 mins exposure to citric acid, samples treated with the nanoparticle suspension softened directionally less than those treated with 300 ppm fluoride.

Scanning electron microscopy (SEM) of polished human enamel incubated in 2.5% w/v aqueous suspensions of nanoparticluate titanium dioxide for 2 mins showed extensive surface coverage of the enamel with inorganic debris. In contrast, SEM images of enamel incubated in 2.5% w/v suspensions of standard micron-sized titanium dioxide showed very little material on the surface of the tissue.

The human enamel specimens were then exposed to 1.0% w/w citric acid, pH 3.8, for 30 mins before re-examination of the surface by SEM. The surface of enamel treated with the nanoparticle suspension was smooth, and polishing lines were clearly visible. Enamel treated with water exhibited the honeycomb pattern indicative of exposed enamel rods visible in surface etched and demineralised enamel.

Energy dispersive X-ray Analysis (EDX) performed on the surface of the enamel confirmed the presence of titanium and oxygen in the sample treated with the nanoparticulate suspension, along with calcium and phosphorous from the enamel mineral itself. The EDX spectrum of enamel treated with standard titanium dioxide showed no evidence of titanium.

The in vitro microhardness study has shown that treatment with titanium dioxide surface-coated with glycerin (with a mean particle size of 20 nm) as a 2.5% w/v aqueous suspension protects against citric acid-induced softening of human enamel. The effect is statistically superior to that seen for treatment with 300 ppm fluoride after 10 mins acid exposure, and equivalent or directionally superior at later time points. In addition, enamel treated with the nanoparticle suspension has been shown to retain a significant surface-coating of titanium dioxide after washing, which inhibits citric acid-induced demineralisation of the tissue surface.

Further microhardness studies, performed using the same methodology as described above, have shown that treatment with nanoparticulate titanium dioxide surface-coated with glycerin (mean particle size 20 nm), as a 2.5% w/v aqueous suspension protects against citric acid induced softening of human enamel to a greater extent than titanium dioxide surface-coated with PVP, stearic acid or uncoated titanium dioxide nanoparticle suspensions (as summarised in FIG. 2). However, uncoated titanium dioxide nanoparticles and titanium dioxide nanoparticles surface-coated with PVP did offer similar protection against the citric acid challenge to treatment with 300 ppm fluoride (positive control).

The treatments tested in this study were: 2.5% w/v titanium dioxide aqueous suspensions, specifically 20 nm glycerin-coated titanium dioxide (UV Titan M212, Kemira, Aston Chemicals), 20 nm PVP-coated titanium dioxide (UV Titan M263, Kemira, Aston Chemicals), 17 nm Stearic acid-coated titanium dioxide (UV Titan M160, Kemira, Aston Chemicals) and 14 nm uncoated titanium dioxide (UV Titan X140, Kemira, Aston Chemicals). A glycerin only negative control was used in the study.

### EXAMPLE 2

### Rehardening of Enamel Erosive Lesions.

Artificial erosive lesions were prepared from polished human enamel mounted in acrylic resin. The lesions were prepared by contacting the mounted specimens for 30 minutes in 10 ml of 1.0% w/w solution of citric acid, pH 3.75. The baseline hardness of each eroded specimen was determined using a Struers Duramin Microindentor, fitted with a Vickers diamond indenter. Hardness values were expressed as Vickers Hardness Numbers (VHN). A load of 1.961N was applied to the specimens, with a dwell time of 20 seconds. The specimens were then randomised and divided into 4 treatment groups (n=6).

Six enamel specimens were placed into one of 3 agitated aqueous nanoparticulate TiO₂ suspensions for 120 seconds, and into a water control. The nanoparticulates tested were 2.5% w/v titanium dioxide (14 nm, UV Titan X140, Lot: 0417002, Kemira, Aston Chemicals), 2.5% w/v glycerin-coated titanium dioxide (20 nm UV Titan M212, Lot: 0132004, Kemira, Aston Chemicals), 2.5% w/v PVP-coated titanium dioxide (20 nm UV Titan M263, Lot: 0339001, Kemira, Aston Chemicals).

The specimens were then removed, washed with deionised water, and placed in 10 ml of a solution containing 300 ppm sodium fluoride for a further 120 seconds. After a further washing step, the enamel was incubated in mucin-free artificial saliva containing 0.02 ppm fluoride. Numerous investigations have shown that resting plaque and saliva contain fluoride in the range 0.02-0.04 ppm. The addition of sodium fluoride to the artificial saliva at a concentration of 0.02 ppm was performed in order to mimic in vivo carryover of fluoride from regular toothpaste brushing.

The enamel was treated first with the nanoparticle suspensions, and then with the sodium fluoride solution. This provides the titanium dioxide particles with the highest potential to affect fluoride uptake, and thus rehardening of the enamel. Specimen rehardening was determined using microindentation at 4 hrs, 24 hrs and 48 hrs. Six indents were obtained for each specimen at each time point.

### RESULTS

The results of the rehardening study are summarised in FIG. 3. The values for enamel hardness have been normalised relative to those obtained after acid softening of the enamel. The data at subsequent time points thus reflects rehardening of the enamel. The error bars in FIG. 3 represent standard deviations.

All the enamel specimens rehardened during the period in which they were exposed to artificial saliva containing 0.02 ppm fluoride. There was no statistically significant difference between any of the treatments and the positive control in this experiment, based on standard deviations.

The in vitro microhardness rehardening study has shown that treatment with 2.5% w/v aqueous suspensions of nanoparticulate titanium dioxide, surface-coated with glycerin, PVP, or uncoated, is not detrimental to the fluoride-induced rehardening of citric acid softened human enamel in vitro.

1-6. (canceled)

7. An oral care composition comprising nanoparticulate titanium dioxide surface-coated with a polyol or polyvinylpyrrolidone (PVP) or a derivative thereof and an orally acceptable carrier or excipient.

8. A composition according to claim 7 wherein the surface-coating material is a polyol selected from the group consisting of glycerin, propylene glycol, polyethylene glycol, polyvinyl alcohol, sorbitol, mannitol or xylitol or a mixture thereof.

9. A composition according to claim 8 wherein the surface-coating material is PVP, a VP/VA copolymer or VP/VOH copolymer, or a mixture thereof.

10. A composition according to claim 7 wherein the surface-coating material is glycerin or propylene glycol.

11. A composition according to claim 7 wherein the surface-coating material is PVP.

12. A composition according to claim 7 wherein the mean particle diameter of the surface-coated nanoparticulate titanium dioxide is from 2 to 500 nm.

13. A composition according to claim 12 wherein the mean particle diameter of the surface-coated nanoparticulate titanium dioxide is from 5 to 250 nm.

14. A composition according to claim 7 wherein surface-coated nanoparticulate titanium dioxide is present in amount from 0.25 to 20% w/w.

15. A composition according to claim 7 further comprising a source of fluoride ions.

16. A composition according to claim 7 further comprising a desensitising agent.

17. A composition according to claim 16 wherein the desensitising agent is a strontium salt or a potassium salt.

18. A dentifrice composition comprising nanoparticulate titanium dioxide as defined in any one of the preceding claims, a fluoride ion source and a silica dental abrasive, the dentifrice having a Relative Dentine Abrasivity (RDA) value from 20 to 60 and a pH in the range 6.5 to 7.5 and being free of an orthophosphate buffer or a water-soluble salt of a C₁₀₋₁₈ alkyl sulphate.

19. A dentifrice composition comprising nanoparticulate titanium dioxide as defined in any one of the preceding claims, a fluoride ion source, a thickening system comprising a thickening silica in combination with xanthan gum optionally with carrageenan and/or a carbomer, an anionic surfactant, and a silica dental abrasive in an amount up to 20% by weight of the total composition, the dentifrice having a pH in the range from 6.0 to 8.0, and being free of an orthophosphate buffer or a calcium salt.

20. A method of combating dental erosion and/or tooth wear which comprises applying an effective amount of a composition comprising nanoparticulate titanium dioxide as defined in claim 7 to an individual in need thereof.
